(19) United States
(12) Patent Application Publication
(10) Pub. No.: US 2024/0197728 A1
(43) Pub. Date: Jun. 20, 2024

(54) METHODS AND PHARMACEUTICAL COMPOSITIONS FOR TREATING REFRACTORY EPILEPSY

(71) Applicants: INSERM (INSTITUT NATIONAL DE LA SANTÉ ET DE LA RECHERCHE MÉDICALE), Paris (FR); SORBONNE UNIVERSITÉ, Paris (FR); CENTRE HOSPITALIER UNIVERSITAIRE DE ROUEN, Rouen (FR)

(72) Inventors: Jean-Christophe PONCER, Paris (FR); Florian DONNEGER, PARIS (FR); Adrien ZANAIN, PARIS (FR)

(21) Appl. No.: 18/555,425

(22) PCT Filed: Apr. 13, 2022
(86) PCT No.: PCT/EP2022/059833

§ 371 Date: Oct. 13, 2023

(30) Foreign Application Priority Data
Apr. 14, 2021 (FR) 21305486.9

(51) Int. Cl. A61K 31/501; A61K 31/5415; A61P 25/08

(52) U.S. Cl. CPC A61K 31/501; A61K 31/5415; A61P 25/08

(57) ABSTRACT

Cation chloride cotransporters (CCC) play a critical role in neuronal chloride homeostasis. Altered CCC expression and function has emerged as a hallmark of wide range of psychiatric and neurological conditions, including various forms of epilepsy. Elevated intraneuronal chloride concentration is thought to result in depolarizing GABA signaling that may contribute to pathological activities and seizures. Compensating for the dysregulation of CCC function in the pathology therefore appears as a promising therapeutic strategy. Bumetanide, an antagonist of the Na/K/Cl co-transporter NKCC1 failed to prevent acute neonatal seizures in the NEMO trial. Here, instead, the inventors tested the effects of novel candidate KCC2 enhancers on epileptiform activity in vitro and in vivo. The inventors show that FDA-approved prochlorperazine (PCPZ) as well as CLP257 potentiate KCC2 function by promoting its membrane clustering, through a mechanism/pathway that does not involve phosphorylation of canonical residues. Both PCPZ and CLP257 reduce interictal activity recorded in vitro in epileptogenic postoperative brain samples from mesial temporal lobe epilepsy patients. In addition, chronic PCPZ administration strongly reduces seizure occurrence in a mouse model of temporal lobe epilepsy. Their results demonstrate for the first time the antiepileptic potential of a KCC2 enhancer and suggest PCPZ may be used in adjunctive therapy in pharmaco-resistant epilepsy.

### FIELD OF THE INVENTION

The present invention relates to the pharmaceutical field. More particularly, the invention relates to use of a KCC2 activator in the treatment of pharmaco-resistant (also known as medically intractable or refractory) epilepsy.

### BACKGROUND OF THE INVENTION

Epilepsy refers to one of the most frequent neurological disorders, affecting over 70 million people worldwide. It is a chronic brain disease characterized by a lasting predisposition to undergo spontaneous and usually unpredictable seizures, with multiple risk factors and a strong genetic predisposition [1]. Currently, epilepsy treatment relies mostly on pharmacology using so-called anti-epileptic drugs (AEDs), resective brain surgery, neurostimulation and dietary therapies. Antiseizure medication controls seizures in about 70% patients but does not improve long-term prognosis. The remaining 30% epilepsy patients develop drug-resistant epilepsy, defined as the failure of at least two, appropriately selected and well-tolerated AEDs to control seizures [2]. Pharmaco-resistant focal epilepsy—such as temporal lobe epilepsy, focal cortical dysplasia or epilepsy associated with low-grade gliomas—may be an indication for resective surgery, with a fairly high rate (≅70%) of seizure control in these patients. However, many pharmaco-resistant epilepsy patients are not eligible to surgery and about 10% may suffer of cognitive sequels. Pharmaco-resistant epilepsy therefore represents a major public health issue as it is associated with severe comorbidities, reduced life quality and increased mortality.

Several AEDs act to potentiate neuronal inhibition mediated by the neurotransmitter GABA, either by increasing GABA release, reducing its catabolism or directly potentiating GABAA receptors which mediate its fast synaptic effects in the brain [2]. GABAA receptors are chloride permeable receptors, the action of which therefore depends on transmembrane chloride gradients. In neurons, these gradients are controlled by cation-chloride cotransporters (CCCs) that maintain low intracellular chloride concentrations and thereby promote a hyperpolarizing and inhibitory chloride influx during GABAA receptors activation [3]. Several forms of pharmaco-resistant epilepsies have been associated with altered expression and function of CCCs [4]. Elevated intraneuronal chloride concentration is thought to result in depolarizing GABAergic signaling that may result in the abnormal neuronal activation and synchronization that underlies seizures. Compensating for the dysregulation of CCC function in the pathology, by maintaining low intracellular chloride concentration, therefore appears as a promising therapeutic strategy. The antagonist of the Na/K/Cl co-transporter NKCC1 bumetanide failed to prevent acute neonatal seizures in the NEMO trial and induced several side-effects due to the broad peripheral expression and functions of NKCC1[5]. Instead, acting on the function of KCC2, a neuron-specific chloride potassium cotransporter, may help restoring normal neuronal chloride transport in the pathology. Thus, increasing the expression or function of KCC2 is expected to restore GABAAR-mediated inhibition and prevent seizures. Recently, screening of compound libraries identified several molecules that may act as KCC2 enhancers [6-8] even though their mechanism of action or even their selective effect on KCC2 remain elusive [7] or disputed [9,10]. In addition, no experimental evidence currently exists demonstrating such compounds would control seizure occurrence in pharmaco-resistant epilepsy.

The present invention has for purpose to identify ways to rescue neuronal chloride transport and control seizures in pharmaco-resistant epilepsy. Herein the inventors have tested the effects of novel candidate KCC2 enhancers in vitro and have shown that their administration strongly reduces epileptiform activity in vitro and seizure occurrence in a mouse model of temporal lobe epilepsy.

### SUMMARY OF THE INVENTION

The present invention relates to using a KCC2 activator in the treatment of refractory epilepsy. The invention also relates to prochlorperazine (PCPZ) and CLP257 or derivatives for use in the treatment of epilepsy. More particularly, the invention is defined by its claims.

### DETAILED DESCRIPTION OF THE INVENTION

Herein the inventors tested the effects of two candidate KCC2 enhancers on epileptiform activity in vitro and in vivo. They demonstrate that FDA-approved prochlorperazine (PCPZ), like CLP257 (a previously reported candidate KCC2 enhancer), potentiates KCC2 function by promoting its membrane clustering and reducing its lateral diffusion. This effect does not appear to involve phosphorylation of previously known KCC2 residues or the WNK/SPAK pathway. They show that both PCPZ and CLP257 reduce interictal activity recorded in vitro from epileptogenic postoperative brain samples from mesial temporal lobe epilepsy patients. In addition, chronic PCPZ administration strongly reduces seizure occurrence in a mouse model of temporal lobe epilepsy. These results demonstrate for the first time the antiepileptic potential of a KCC2 enhancer and suggest PCPZ may be used in adjunctive therapy in pharmaco-resistant epilepsy.

### Therapeutic Methods and Uses

Accordingly, in a first aspect the present invention relates to a method for treating refractory epilepsy in a subject in need thereof comprising administering an effective amount of a KCC2 activator.

In other words, the invention relates to a KCC2 activator for use for treating refractory epilepsy in a subject in need thereof.

As used herein, the term “epilepsy” refers to one of the most common serious brain conditions, affecting over 70 million people worldwide. Epilepsy is a chronic brain disorder characterized by recurring, unprovoked seizures. A person is diagnosed with epilepsy upon occurrence of at least two unprovoked seizures (i.e. not caused by known and reversible medical condition). Epilepsy can be classified according electroclinical characteristics, following the Classification and Terminology of the International League Against Epilepsy (ILAE). Epilepsy comprises both generalized and focal forms, with generalized epilepsy affecting both hemispheres while focal epilepsy includes unifocal and multifocal disorders as well as seizures involving one hemisphere.

According to the invention, epilepsy includes but is not limited to benign familial epilepsy, benign infantile epilepsy, early myoclonic encephalopathy (EME), mycolonic encephalopathy, Myoclonic epilepsy in infancy (MEI), Ohtahara syndrome, Febrile seizures plus (FS+), Panayiotopoulos syndrome, Epilepsy with myoclonic atonic (previously astatic) seizures, Benign epilepsy with centrotemporal spikes (BECTS), Autosomal-dominant nocturnal frontal lobe epilepsy (ADNFLE), Late onset childhood occipital epilepsy (Gastaut type), Epilepsy with myoclonic absences, Lennox-Gastaut syndrome, Epileptic encephalopathy with continuous spike-and-wave during sleep (CSWS), Landau-Kleffner syndrome (LKS), Childhood absence epilepsy (CAE), Juvenile absence epilepsy (JAE) Juvenile myoclonic epilepsy (JWE), Epilepsy with generalized tonic-clonic seizures alone, Progressive myoclonus epilepsies (PME), Autosomal dominant epilepsy with auditory features (ADEAF), Mesial Temporal Lobe Epilepsy (MTLE), Rasmussen syndrome, Gelastic seizures with hypothalamic hamartoma, Hemiconvulsion-hemiplegia-epilepsy benign Rolandic epilepsy, a frontal lobe epilepsy, an infantile spasms, a juvenile myoclonic epilepsy, a juvenile absence epilepsy, a childhood absence epilepsy (pyknolepsy), a hot water epilepsy, a Lennox-Gastaut syndrome, a Landau-Kleffner syndrome, a Dravet syndrome, a progressive myoclonus epilepsies, a reflex epilepsy, a Rasmussen's syndrome, a temporal lobe epilepsy, a limbic epilepsy, a status epilepticus, an abdominal epilepsy, a massive bilateral myoclonus, a catamenial epilepsy, a Jacksonian seizure disorder, a Lafora disease or photosensitive epilepsy.

As used herein the term “refractory epilepsy”, also known as “treatment-resistant epilepsy” (TRE), “drug-resistant epilepsy” or “pharmaco-resistant epilepsy”, affects 30% of epilepsy patients and is associated with severe morbidity and increased mortality. Refractory epilepsy arises from a failure to achieve persistent seizure remission after trials of at least two, appropriately selected antiepileptic drug (AED) regimens that are tolerated at therapeutic dosages [8]. All epilepsies can be pharmaco-resistant, although seizures associated with epileptic encephalopathies (e.g. Dravet Syndrome (DS) and Lennox-Gastaut Syndrome (LGS)), Febrile Infection-Related Epilepsy Syndrome (FIRES) and epilepsy associated with Tuberous Sclerosis Complex (TSC) are among the most refractory to medical therapies [9].

In some embodiment, the refractory epilepsy is a refractory temporal lobe epilepsy.

As used herein the term “Temporal Lobe Epilepsy” or “TLE” denotes a chronic neurological condition characterized by chronic and recurrent seizures (epilepsy) which originate in the temporal lobe of the brain.

As used herein, the term “subject” refers to any mammals, such as a rodent, a feline, a canine, and a primate. Particularly, in the present invention, the subject is a human afflicted with or susceptible to be afflicted with epilepsy, and in particular refractory epilepsy.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. According to the invention, the KCC2 activator allows to ameliorate the symptoms of seizures and epileptiform discharges.

The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein the terms “administering” or “administration” refer to the act of injecting or otherwise physically delivering a substance as it exists outside the body (e.g. a KCC2 activator) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

In some embodiment, the KCC2 activator is chronically administrated to the subject.

As used herein, the term “chronic” or “chronically” administration refers to an administration of KCC2 activator that is persistent or otherwise long-lasting. In some embodiment, the chronic administration is a continuous administration of the KCC2 activator for at least 5 to 30 days, at least 1 month, at least 3 months, at least 1 year, or more preferably as long as the subject will need it (such as, for example, if any timeout of the treatment leads to the reappearance of the symptoms of the disease). In some embodiment, the chronic administration is a twice-daily administration. In some embodiment, the chronic administration is 1, 2, 3 or 4 daily administration of the substance.

By a “therapeutically effective amount” is meant a sufficient amount of KCC2 activator of the invention for use in a method for the treatment of refractory epilepsy at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

As used herein, the term “KCC2”, also known as “SLC12A5” or “potassium-chloride transporter member 5” has its general meaning in the art and refers to a member of the solute carrier 12 (SLC12) family encoded by SLC12A5 gene and that is selectively expressed in mature neurons. It is a critical mediator of synaptic inhibition, cellular protection against excitotoxicity and may also act as a modulator of neuroplasticity. KCC2 is a neuron-specific potassium-chloride cotransporter that controls transmembrane chloride gradients, and thus maintaining low intracellular chloride. It is well established that sustaining an inwardly-directed electrochemical Cl— gradient across the neuronal plasma membrane is critical for a proper, inhibitory function of postsynaptic GABAA receptor signaling

As used herein, the term “KCC2 activator” has its general meaning in the art and refers to any compound that is able to activate or increase the activity or expression of KCC2.

As used herein, the term “activity of KCC2” refers to the net transport activity of KCC2 and in particular to the maintaining of an inwardly-directed electrochemical Cl— gradient across the neuronal plasma membrane. In some embodiments, the KCC2 activator may promote KCC2 membrane clustering. In particular embodiments, the KCC2 activator may promote KCC2 membrane clustering by acting on mechanisms that do not necessarily involve a change in the phosphorylation state of the transporter. In some embodiment, the KCC2 activator of the present invention may act to directly activate or increase the intrinsic activity, membrane stability or expression of KCC2.

Tests for determining the capacity of a compound to be an activator of KCC2 are well known to the person skilled in the art. In a preferred embodiment, the KCC2 activator acts either on KCC2 itself or via a regulatory or interacting protein to increase the biological activity of KCC2. Activators of KCC2 may be determined by any competing assay well known in the art. For example, the assay may consist in determining the ability of the agent to promote KCC2 membrane expression, clustering or function, as detected by imaging or electrophysiological means. The binding ability is reflected by the KD measurement. The term “KD”, as used herein, is intended to refer to the dissociation constant, which is obtained from the ratio of Kd to Ka (i.e. Kd/Ka) and is expressed as a molar concentration (M). KD values for binding biomolecules can be determined using methods well established in the art. In specific embodiments, an KCC2 activator that “specifically” binds to KCC2 or a regulatory protein is intended to refer to an activator that binds to human KCC2 receptor or a regulatory protein with a KD of 1 μM or less, 100 nM or less, 10 nM or less, or 3 nM or less. Then a competitive assay may be settled to determine the ability of the agent to enhance the biological activity of KCC2. The functional assays may be envisaged such as evaluating the ability i) to reduce KCC2 membrane diffusion and to promote its clustering in neurons, ii) to promote transmembrane chloride extrusion, as detected electrophysiologically and/or iii) to prevent epileptiform activity in vitro or in vivo (see example and FIG. 2-5);

The skilled in the art can easily determine whether a KCC2 activator enhances, increases or activates the biological activity of KCC2.

For instance, the reduction of KCC2 membrane diffusion can be measured by using quantum dot-based single particle tracking, or by using immunostaining of KCC2 (see example). Epileptiform activity assay is also well known in the art and described in various publications.

The increase of the KCC2 activity and/or expression may be determined by measuring the expression level of SLC12A5 gene and/or KCC2 protein in neurons treated with KCC2 activator.

The expression level of mRNA may be determined by any suitable methods known by skilled persons. For example, the nucleic acid contained in the sample is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR).

The level of the KCC2 protein may also be determined by any suitable methods known by skilled persons. The quantity of the protein may be measured, for example, by semi-quantitative Western blots, enzyme-labelled and mediated immunoassays, such as ELISAs, biotin/avidin type assays, radioimmunoassay, immunoelectrophoresis, mass spectrometry, or immunoprecipitation or by protein or antibody arrays.

The expression and/or activity of KCC2 can be increased by agents including, but are not limited to, chemical compounds, compounds known to modify gene expression, modified or unmodified polynucleotides (including oligonucleotides), polypeptides, peptides, small RNA molecules and miRNAs. Such agents are well-known in the art.

In some embodiment, the KCC2 activators are selected from the group consisting of prochlorperazine, CLP257, CLP290, or CLP257 derivatives, or CLP290 derivatives.

In some embodiment, the KCC2 activator is CLP257.

As used herein, the term “CLP257” also known as “(5Z)-5-[(4-Fluoro-2-hydroxyphenyl)methylene]-2-(tetrahydro-1-(2H)-pyridazinyl)-4(5H)-thiazolone” has its general meaning in the art and refers to compound with the following formula (C₁₄H₁₄FN₃O₂S).

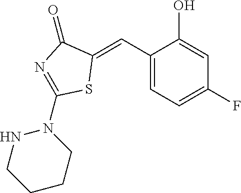 [SMILES: O=C1N=C(N2CCCCN2)S/C1=C\c1ccc(F)cc1O]

Its CAS number is 1181081-71-9.

In some embodiment, the KCC2 activator is CLP290.

As used herein, the term “CLP290” also known as “[5-Fluoro-2-[(Z)-(2-hexahydropyridazin-1-yl-4-oxo-thiazol-5-ylidene)methyl]phenyl] pyrrolidine-1-carboxylate” has its general meaning in the art and refers to compound with the following formula (C₁₉H₂₁FN₄O₃S):

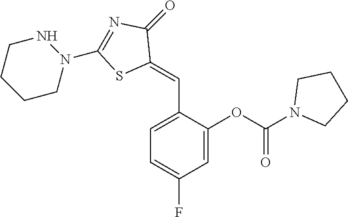 [SMILES: O=C1N=C(N2CCCCN2)S/C1=C\c1ccc(F)cc1OC(=O)N1CCCC1]

Its CAS number is 1181083-81-7.

As used herein, the term “CLP257 derivatives” and “CLP290 derivatives” has its general meaning in the art and refers to compounds derived from CLP257 and CLP290. CLP257 or CLP290 derivatives possess the desired pharmacological activity of CLP257 or CLP290, i.e. is capable to enhance KCC2 activity. In particular embodiment CLP257 or CLP290 derivatives refers to arylmethylidene heterocycles described in WO2009/097695.

In some embodiment, the KCC2 activator is prochlorperazine.

As used herein, the term “prochlorperazine” (PCPZ), also known as “compazine”, “capazine” or “stemetil” has its general meaning in the art and refers to piperazine phenothiazine with the following formula (C₂₀H₂₄ClN₃S).

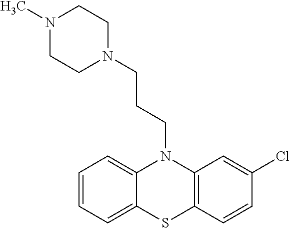 [SMILES: CN1CCN(CCCN2c3ccccc3Sc3ccc(Cl)cc32)CC1]

PCPZ is a first-generation antipsychotic drug that is used for the treatment of severe nausea and vomiting, as well as short-term management of psychotic disorders such as generalized non-psychotic anxiety and schizophrenia. Prochlorperazine was approved for medical use in the United States in 1956 and is available as a generic medication. Its CAS number is 58-38-8.

As used herein, the term “pharmaceutically” or “pharmaceutically acceptable” refers to molecular entities and compositions that do not produce an adverse, allergic or other untoward reaction when administered to a mammal, especially a human, as appropriate.

The KCC2 activator can be administered in combination with anti-epileptic compounds.

As used herein, the term “anti-epileptic drugs” refers to compounds well known in the art and used to treat epilepsy. In the context of the invention, anti-epileptic compounds include but are not limited to sodium valproate; acetazolamide; adrenocorticptrophin, benzodiazepines such as clorazepate, clobazam, clonazepam, clotiazepam, diazepam, oxazepam, alprazolam, lorazepam, bromazepam, prazepam, nordazepam, loflazepate, acepromazine, nitrazepam, midazolam, lormetazepam, flunitrazepam, temazepam, loprazolam, estalzolam, zolpidem, zopiclone, carbamazepine; ethosuximide; felbamate, gabapentin; lacosamide; lamotrigine; levetiracetam; oxcarbazepine; paraldehyde; barbiturate compounds such as primidone, mephobarbital, thiopental and methohexital; phenobarbital; phenytoin; potassium bromide; stiripentol; sulthiame; tiagabine; topiramate; pregabalin; vigabatrin and zonisamide

In a particular embodiment, the invention relates to i) a KCC2 activator and ii) anti-epileptic compound used as a combined preparation for use in the treatment of refractory epilepsy.

In a particular embodiment, i) a KCC2 activator and ii) anti-epileptic compound as a combined preparation according to the invention for simultaneous, separate or sequential use in the use in the treatment of refractory epilepsy.

In another aspect of the invention, the present invention relates to a method for treating epilepsy in a subject in need thereof comprising administering an effective amount of prochlorperazine (PCPZ), CLP290, CLP257 or CLP257 derivatives, or CLP290 derivatives.

In a particular embodiment, PCPZ, CLP290, CLP257 or CLP257 derivatives, or CLP290 derivatives, is administered in combination with anti-epileptic compounds.

In some embodiment, the present invention relates to a method for treating epilepsy in a subject in need thereof comprising administering an effective amount of prochlorperazine (PCPZ).

In a particular embodiment, the epilepsy is a refractory epilepsy.

In some embodiment, the refractory epilepsy is a refractory temporal lobe epilepsy.

In some embodiment, the prochlorperazine is chronically administrated to the subject.

### Pharmaceutical Composition

The KCC2 activator for use of the invention may be used or prepared in a pharmaceutical composition.

In one embodiment, the invention relates to a pharmaceutical composition comprising the KCC2 activator for use in the treatment of refractory epilepsy in a subject of need thereof.

In some embodiment, the refractory epilepsy is refractory TLE.

In some embodiment, the KCC2 activator is prochlorperazine (PCPZ), CLP290, CLP257, or CLP257 derivatives, or CLP290 derivatives.

In some embodiment, the KCC2 activator is prochlorperazine (PCPZ).

The invention also relates to a pharmaceutical composition comprising PCPZ, CLP257, CLP257, or CLP290 derivatives, or CLP290 derivatives for use in the treatment of epilepsy in a subject of need thereof.

Typically, the KCC2 activator, and in particular PCPZ, may be combined with pharmaceutically acceptable excipients, and optionally sustained-release matrices, such as biodegradable polymers, to form therapeutic compositions.

A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising inhibitors of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The inhibitor of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1. PCPZ and CLP257 promote chloride extrusion in hippocampal neurons. A. Representative currents evoked at varying potentials by focal application of isoguvacine (arrow, 100 μM) onto the soma or dendrite of hippocampal neurons (DIV 21-24) previously treated for 2 h with either vehicle only (DMSO, control) or PCPZ (10 μM). B. I-V curves corresponding to the traces shown in A. C. Summary graphs showing reversal potential (top, E_{GABA}) and somatodendritic E_{GABA} gradient (bottom, ΔE_{GABA}) in control and PCPZ-treated neurons (n=19 neurons and n=16 neurons respectively, from 5 independent cultures). * t-test p<0.05, ** t-test p<0.01. D-F, same as in A-C, with currents evoked at varying potentials by focal uncaging of RubiGABA (arrow, 30 μM) onto the soma or dendrite of hippocampal neurons, upon application of 100 μM CLP257 or DMSO. (n=10 and 8 neurons, respectively, from 4 independent cultures). * t-test p<0.05.

FIG. 2. CLP257 but not PCPZ modulates GABAA receptor function. A. Summary graph showing averaged normalized amplitude of currents evoked by focal somatic application of isoguvacine before and during acute application of 10 μM PCPZ. Inset, representative averaged evoked currents before and during PCPZ application. B. Summary graphs showing the effect of PCPZ on the peak amplitude (left) and decay time constant (ti) of isoguvacine-evoked currents (n=10 neurons each from 2 independent cultures). C. Representative, 5 s mIPSC recording before (DMSO) and during application of PCPZ. Inset, superimposed averaged mIPSCs detected from this recording. D, Summary graphs showing the effect of PCPZ application on the amplitude (left), frequency (middle) and decay time constant (right) of mIPSCs (n=18 neurons each from 4 independent cultures. *** Wilcoxon test, p<0.001). E-H, same as in A-D for currents recorded in the presence of 100 μM CLP257 versus DMSO. E-F, Unlike PCPZ, CLP257 increased the decay time constant of RubiGABA evoked currents with no significant effect of their amplitude. (n=11 neurons each from 2 independent cultures. * ** Wilcoxon test, p<0.001). G-H, CLP257 had no effect on mIPSC amplitude, decay time or frequency. (n=12 neurons each from 1 culture).

FIG. 3. Both PCPZ and CLP257 reduce KCC2 membrane diffusion and promote its clustering in hippocampal neurons. A. Representative immunoblots (left) of protein extracts from hippocampal cultures treated for 2 hours with either PCPZ (10 μM in DMSO), CLP257 (100 μM in DMSO) or DMSO alone. Right, quantification of 3 independent experiments. No significant change in total KCC2 expression was detected upon PCPZ or CLP257 treatment as compared to control (t-test p=0.14 and p=0.89, respectively). B. Immunoblots (left) and quantification (right) showing biotinylated surface KCC2 fraction (bound/total) was also unaffected upon PCPZ or CLP257 treatment. (n=3 independent cultures, t-test p=0.74 and 0.80, respectively). C. Representative recombinant KCC2 extrasynaptic trajectories, as detected using quantum dot-based single particle tracking, showing reduced lateral diffusion upon treatment with PCPZ or CLP257. Scale, 1 μm. D. Logarithmic distributions of median diffusion coefficients (D) and explored areas (EA) of extrasynaptic (extra) or synaptic (syn) KCC2 trajectories, in control neurons (DMSO, D extra n=614, D syn n=162 and EA extra n=1836, EA syn n=486 quantum dots) and neurons treated for 2 hours with PCPZ (D extra n=512, D syn=131 and EA extra n=1536, EA syn n=393 quantum dots) or CLP257 (D extra n=697, D syn n=179 and EA extra n=2091, EA syn n=537 quantum dots). (4 independent cultures. Mann-Whitney test * p<0.05 **p<0.01 ***p<0.001). E. Confocal micrographs showing KCC2 immunostaining in cultured hippocampal neurons treated for 2 hours with PCPZ, CLP257 or DMSO only. Scale, 5 μm. F. Boxplots showing the distributions of integrated intensity, area and density of KCC2 clusters in control neurons and neurons exposed to either PCPZ or CLP257, for 30 minutes or 2 hours. Note that the effects of PCPZ and CLP257 are only apparent at 2 hours but not after 30 minutes only. n=10 neurons per condition per culture and 4 independent cultures per condition except for CLP257-30 min (3 independent cultures). t-test or Mann-Whitney test * p<0.05 **p<0.01 ***p<0.001

FIG. 4. PCPZ and CLP257 do not affect phosphorylation of canonical KCC2 residues. A. Quantification of immunoprecipitated (IP) proteins or protein lysates, prepared from hippocampo-cortical slices treated for 2 hours with either DMSO, CLP257 (100 μM in DMSO), PCPZ (10 μM in DMSO), or the pan-WNK inhibitor WNK463 (10 μM in DMSO). (n=3 independent replicates/rats, t-test *p<0.05 ***p<0.001) B. Quantification of IP proteins, prepared from hippocampo-cortical slices treated for 2 hours with either DMSO, CLP257 (100 μM in DMSO), PCPZ (10 μM in DMSO), or sodium orthovanadate (Na₃VO₄, 200 μM in H₂O). (n=4 independent replicates/rats, Mann-Whitney test *p<0.05). C. kinase activity profile of recombinant human OSR1, STK39 (SPAK), WNK1 and WNK3 in the presence of PCPZ (black symbols) or CLP257 (striped symbols) concentrations ranging 0.1-500 or 1-1000 μM, respectively. Mean values±SD from 2 replicates were normalized to control (DMSO). Open symbols represent PCPZ and CLP257 concentration used in all other in vitro assays (10 and 100 μM, respectively). No significant inhibition of any kinase was observed at these concentrations.

FIG. 5. PCPZ and CLP257 prevent spontaneous interictal-like discharges in temporal lobe slices from mTLE patients. A. Representative local field potential recordings showing spontaneous interictal-like discharges (IIDs) in a slice maintained in control conditions (top) and upon >10 min application of 10 μM bumetanide (bottom). Insets show individual IIDs. B. same as A for two slices from the same patient kept for 2 hours in ACSF supplemented with either 10 μM PCPZ or 100 μM CLP257. The slice treated with PCPZ showed no spontaneous IID. However, application of a pro-convulsant ACSF (6.5 mM K⁺ and 0.25 Mg²⁺) on the same slice induced high spontaneous activity, including IIDs and multiunit activity. Bottom, similar observation on a slice from the same patient, kept for 2 hours in ACSF supplemented with 100 μM CLP257. C. Summary plot showing the proportion of slices with and without IIDs. Chi² test, * p<0.05. D. Graphs showing the mean frequency of spontaneous IIDs (normalized to control) recorded in slices from 7 mTLE patients kept in control ACSF (DMSO) or treated with 10 μM PCPZ. Two groups were distinguished: responders (Group I, 5 patients) and non-responders (Group II, 2 patients). Similar data are shown for slices from 6 patients, kept in control ACSF or treated with 100 μM CLP257. Out of the 6 patients, only one showed no effect of CLP257 on IID frequency. Wilcoxon signed rank test, * ** p<0.001.

FIG. 6. Chronic PCPZ treatment reduces seizure frequency in a mouse model of mesial temporal lobe epilepsy. A. experimental epilepsy paradigm. scopo: scopolamine, pilo: pilocarpine. Status epilepticus (SE) was interrupted one hour after onset with ip injection of ketamine (keta.) and valium. See methods for details. B. typical ECoG recordings before, during and after pilocarpine-induced SE. C. Illustration of typical ECoG signals automatically detected from recordings as in B. D. Graph showing the cumulative number of seizures detected in a mouse immediately after SE and up to 37 days post SE. Recording was interrupted between days 21 and 34 post SE. Note that seizures stop 2 days after SE and chronic seizures occur at a regular rate after day 34 (inset). E. top experimental timeline: mice received two daily saline injections between days 35-40 (before) and saline or PCPZ injections between days 41 and 45 (after). Summary graphs comparing mean daily seizure numbers between days 41 and 45 (after) and days 35 to 40 (before). PCPZ injections (n=8 mice) significantly reduced the frequency of daily seizures (* paired t-test p<0.05) while saline injections (n=7) had no effect (p=0.49).

### EXAMPLE

### Material & Methods

### Primary Hippocampal Cultures

All animal procedures were carried out according to the European Community Council directive of 24 Nov. 1986 (86/609/EEC), the guidelines of the French Ministry of Agriculture and the Direction Departementale de la Protection des Populations de Paris (Institut du Fer i Moulin, license C 72-05-22). All efforts were made to minimize animal suffering and to reduce the number of animals used.

Primary cultures of hippocampal neurons were prepared as previously described [14,17]. Briefly, hippocampi were dissected from embryonic day 18-19 Sprague-Dawley rats of either sex. Tissue was then trypsinized (0.25% v/v), and mechanically dissociated in 1×HBSS (Invitrogen) containing 10 mM HEPES. Neurons were plated at a density of 120×103 cells/ml onto 18-mm diameter glass coverslips (Assistent) pre-coated with 50 μg/ml poly-D,L-ornithine (Sigma-Aldrich) in plating medium composed of Minimum Essential Medium (MEM, Sigma-Aldrich) supplemented with horse serum (10% v/v, Invitrogen), L-glutamine (2 mM) and Na+ pyruvate (1 mM) (Invitrogen). After attachment for 3-4 h, cells were incubated in culture medium composed of Neurobasal medium supplemented with B27 (1×), L-glutamine (2 mM), and antibiotics (penicillin 200 units/ml, streptomycin, 200 μg/ml; Invitrogen) for up to 4 weeks at 37° C. in a 5% CO₂ humidified incubator. Each week, one fifth of the culture medium volume was renewed.

### Pharmacology

Prochlorperazine dimaleate (Sigma-Aldrich) stock was prepared at 25 mM in dimethyl sulfoxide (DMSO, Sigma-Aldrich) and used at a final concentration of 10 μM. CLP257 (Tocris) stock was prepared at 10 mM in DMSO and used at a final concentration of 100 μM. WNK463 (Tocris) stock was prepared at 50 mM in DMSO and used at a final concentration of 10 μM. Equimolar DMSO concentration was used as control. Sodium orthovanadate (Sigma-Aldrich) stock was prepared at 100 mM in H₂O and used at a final concentration of 100 μM.

For all experiments on hippocampal cultures, neurons were pre-incubated for 2 hours with either DMSO, PCPZ or CLP257 in a CO₂ incubator set at 37° C. Drugs were added directly to the culture medium. For electrophysiology experiments, neurons were also recorded in presence of these drugs in the bath. For biochemistry experiments on rat hippocampo-cortical slices, slices were incubated with either DMSO (vehicle), CLP257, PCPZ, WNK463 or sodium orthovanadate for 2 hours in interface chambers set at 37° C. For human tissue experiments, slices were pre-incubated with either DMSO, CLP257 or PCPZ for 2 hours in interface chambers set at 37° C. and then also recorded in presence of these compounds.

### Cellular Electrophysiology

Neurons were maintained at 33° C. in an extracellular medium containing (in mM): NaCl 120, D-glucose 20, HEPES 10, MgCl₂ 3, KCl 2, CaCl₂ 2 (pH 7.4), in a recording chamber (Luigs & Neumann) equipping an upright microscope (BX51WI; Olympus). Neurons were patch clamped in whole-cell configuration, with a borosilicate glass pipette containing (in mM): K-gluconate 104, KCl 25.4, HEPES 10, EGTA 10, MgATP 2, Na₃GTP 0.4 and MgCl₂ 1.8 (pH 7.4) and held at −65 mV. GABAergic currents were induced at the somatic or dendritic level (approx. 50-80 μm from the soma) by focal application of isoguvacine (100 μM, Sigma-Aldrich) through a second pipette connected to a PicoSpritzer set at 10 psi for 10 (soma) or 100 (dendrites) ms. In some experiments, GABAergic currents were induced by locally uncaging RubiGABA (30 μM) using a laser pulse (405 nM, Omicron Deepstar) of 1-8 ms and 15-80 mW delivered using a photolysis head (Prairie Technologies) through the objective [16]. Neurons were voltage clamped from −85 to −5 mV with 3.5 s step increments of 10 mV. The current-voltage relation of somatic and dendritic currents was then calculated from the peak amplitude of GABAergic currents recorded at each potential, corrected for the liquid junction potential (−15.2 mV in our conditions) and voltage drop across the access resistance.

### Surface Biotinylation

Neurons were washed with ice-cold PBS three times, and then incubated with PBS supplemented with 0.5-1 mg/ml EZ-Link Sulfo-NHS-SS-Biotin (Pierce, Rockford, IL, USA) at 4° C. for 30 min under gentle agitation. Biotinylation was stopped by addition of Tris-HCl (50 mM; pH 7.4) and cells lysed in a modified RIPA buffer containing (in mM): Tris-HCl 50 (pH 7.4), NaCl 150, 1% Nonidet P-40, 0.5% DOC, 0.1% SDS, NaF 50, Na3VO4 1 and protease and phosphatase inhibitors (Roche). Lysates were then collected by gently scraping the bottom of each well. After complete homogenization, samples were centrifuged and the supernatant was collected. A small fraction of the lysates was kept for input KCC2 and total protein quantification using BCA kit (Thermo Scientific). Lysates were mixed with a 50% slurry of Neutravidin beads (Thermo Scientific) and rotated overnight at 4° C. Beads were then pelleted by centrifugation and the supernatant (non-biotinylated fraction) was collected. Beads were then washed three times in modified RIPA buffer and one time in modified RIPA buffer without detergents. After the last wash, the biotinylated fraction was carefully removed from the beads. Total, biotinylated and non-biotinylated fractions were then denaturated in 10% 3-mercaptoethanol and negatively charged in 6×SDS sample buffer at 37° C. for one hour.

Samples were subjected to electrophoresis on polyacrylamide gradient gels (4-12%) and transferred to nitrocellulose membranes. The transfer efficiency was checked by addition of red Ponceau. Membranes were washed for 30 min with TBS-tween solution (TBST; Invitrogen) and blocked with a solution containing 5% (w/v) skim milk. Membranes were then incubated with a rabbit primary antibody against KCC2 (07-432; Millipore; 1:3000) and a mouse primary antibody against neuron-specific beta-III tubulin (clone TuJ-1; MAB1195; R&D System; 1:1000) overnight at 4° C. They were then washed three times with a TBST-milk solution and incubated for one hour at RT with goat secondary antibodies anti-rabbit DyLight 800 (1:3000; Rockland) and anti-mouse DyLight 700 (1:1000; Rockland). Membranes were then washed again with TBST three times and one time with TBS. Fluorescence was detected using Odyssey infrared imaging system (LI-COR Bioscience). The relative intensities of immunoblot bands were determined by densitometry using ImageJ software. Total KCC2 protein expression was determined as the sum of monomeric and oligomeric bands normalized to tubulin. Surface expression of KCC2 was determined as the ratio of monomeric+oligomeric biotinylated KCC2 fraction vs. total KCC2 fraction (biotinylated fraction+non-biotinylated fraction).

### DNA Constructs and Transfection

For single-particle-tracking experiments, a previously described KCC2-Flag construct was used [17]. This recombinant Flag-tagged KCC2 transporter was shown to retain normal traffic and function in transfected hippocampal neurons. All constructs were sequenced by Beckman Coulter Genomics (Hope End, Takeley, U.K). Neuronal transfections were performed at DIV 13-14 using Transfectin (BioRad), according to manufacturer's instructions (DNA:transfectin ratio 1 μg:3 μl), with 1-1.5 μg of plasmid DNA per 20 mm well. Experiments were performed 7-10 days post-transfection.

### Immunocytochemistry

Neurons were fixed for 15 min at room temperature (RT), with paraformaldehyde (PFA, 4% w/v in PBS). Cells were then washed in PBS, permeabilized with 10% Triton X100 for 4 min at RT and then exposed to a blocking solution containing goat serum (20% v/v; Invitrogen) and 0.1% Triton X100 diluted in PBS. Neurons were incubated for 1 hour at RT with rabbit KCC2 antibody (1: 500; Sigma) and mouse MAP2 antibody (1:500; Chemicon). Neurons were then incubated for 45 min at RT with AlexaFluor 488-conjugated goat anti-mouse antibody (1:400; Jackson Labs) and with Cy3-conjugated goat anti-rabbit antibody (1:400; Jackson Labs). Neurons were then labeled with DAPI (1:2000, 5 min), washed in PBS and mounted on slides with Mowiol 844 (48 mg/ml, Sigma).

### Fluorescence Image Acquisition and Analysis

Images were acquired using a 63×objective (NA 1.40) mounted on an upright epifluorescence microscope (DM6000, Leica) equipped with a 12-bit cooled CCD camera (Micromax, Roper Scientific) operated with MetaMorph software (Roper Scientific). For KCC2 clusters detection, exposure time was adjusted to obtain best fluorescence to noise ratio and to avoid pixel saturation. This exposure time was maintained unchanged among cells and conditions. For experiments on non-transfected cells and for each neuron, dendritic sections were chosen using MAP2 labeling and then an image with tetramethylrhodamine (TRITC) filter was acquired to visualize KCC2 labeling.

KCC2 clusters analysis was performed using MetaMorph software (Roper Scientific), as previously described [17]. Images were first flatten background filtered (kernel size, 3×3×2) to enhance cluster outline and a user-defined intensity threshold was applied to select clusters and avoid their coalescence. Clusters were delineated in the area of interest (AOI) and the corresponding regions were transferred onto raw images to quantify cluster density (mean KCC2 cluster number per 10 μm²) and integrated cluster intensity (integrated fluorescence pixel intensity within clusters). For each culture, the analysis was done on 10 cells per condition and approx. 100 clusters per cell.

### Single-Particle Tracking and Analysis

Neurons were stained as previously described [17]. Briefly, cells were incubated for 6 min at 37° C. with a mouse anti-Flag antibody (mouse, 1:300, Sigma-Aldrich), washed and incubated for 6 min at 37° C. with a biotinylated Fab goat anti-mouse antibody (1:300; Jackson Immuno Research) in imaging medium. After washes, cells were incubated for 1 min with streptavidin-coated quantum dots (QDs) emitting at 655 nm (1 nM; Invitrogen) supplemented with PBS (1 M; Invitrogen) and 10% Casein (v/v) (Sigma).

Cells were imaged using an Olympus IX71 inverted microscope equipped with a 60× objective (NA 1.42; Olympus) and a 120 W Mercury lamp (X-Cite 120Q, Lumen Dynamics). QD real time recordings (integration time of 30 ms over 1200 consecutive frames) were acquired with an ImagEM EMCCD camera and MetaView software (Meta Imaging 7.7). Cells were imaged within 45 min following appropriate drugs pre-incubation.

QD tracking and trajectory reconstruction were performed with homemade Matlab routines, as described [17]. For each imaged neuron, one or two dendritic subregions were quantified. Mean square displacement (MSD) over time plots were calculated as described and diffusion coefficients (D) were calculated by fitting the first four points without origin of the MSD vs. time curves with the equation: MSD(nτ)=4Dnτ+b, where b is a constant reflecting the spot localization accuracy. The explored area of each trajectory was defined as the MSD value of the trajectory at two different time intervals of at 0.42 and 0.45 s.

### Immunoprecipitation and Immunoblot

8-weeks old Sprague Dawley male rats were anesthetized by intraperitoneal ketamine/xylazine injection (120/10 mg/kg) and perfused transcardially with ice-cold (0-4° C.), oxygenated solution (O₂/CO₂ 95/5%), containing (in mM): N-methyl-d-glucamine 93, KCl 2.5, NaH2PO4 1.2, NaHCO₃30, HEPES 20, D-glucose 20, ascorbic acid 5, sodium pyruvate 3, MgSO4 10 and CaCl₂) 0.5 (300-310 mOsm, pH7.4). Brains were removed and transverse hippocampo-cortical slices (400 m thick) were prepared in the same solution using a vibratome (HM650V, Microm). Slices were then incubated for 2-hours with DMSO, the KCC2 enhancers (CLP257, PCPZ, WNK43) or sodium orthovanadate in an interface chamber containing artificial cerebrospinal fluid (ACSF) composed of (in mM): D-glucose 10, KCl 3.5, NaHOC3 26, NaH2PO4 1.25, NaCl 126, CaCl₂) 1.6 and MgCl2 1.2 (290 mOsm), equilibrated with 5% CO2 in 95% 02. After the treatments, slices were washed with cold PBS, instantaneously frozen with liquid nitrogen and stored at −80° C.

Slices were mechanically lysed on ice, using a 2 mL Dounce, in a buffer containing (in mM): Tris/HCl, pH 7.5 50, EGTA 1, EDTA 1, sodium orthovanadate 1, sodium-B-glycerophosphate 10, sodium fluoride 50, sodium pyrophosphate 5, sucrose 270, benzamidine 1, phenylmethylsulfonyl fluoride 2, Triton 10%, (v/v) 2-mercaptoethanol 0.1% and protease inhibitors (Roche). After complete homogenization, samples were centrifuged (16,000×g at 4° C. for 10 min) and the supernatant was collected. Protein quantification was done using Pierce BCA Protein Assay kit (Thermo Scientific) with BSA as standard.

The following phosphorylation site-specific antibodies: anti-KCC3A phospho Thr1048 (0.35 mg/ml, sheep, [S0961C] 1st bleed, Dundee), anti-KCC3A phospho Thr 991 (0.26 mg/ml, sheep, [S959C] 1st bleed, Dundee) were first incubated for 30 min at 4° C. with the corresponding non-phosphorylated peptide (10 mg/ml, Dundee).

Phospho-antibodies or anti-KCC2 antibodies (07-432; Millipore) were then coupled with protein-G-Sepharose (4 Fast Flow; Sigma-Aldrich) at a ratio of 15 g of antibody per 100 μl of beads, for 2 h at 4° C. without agitation. 1.5 mg of clarified cell lysate was incubated with the antibody-coupled bead suspension, overnight at 4° C. under gentle agitation. The supernatant was collected after centrifugation and beads were washed twice with NaCl solution (0.25M NaCl in 1×PBS) and twice with PBS alone. Bound proteins were eluted with 2×LDS sample buffer (Invitrogen) containing 0.5% (v/v) 2-mercaptoethanol and denaturated at 75° C. for 20 min (IP with phospho-antibodies) or 37° C. for 1 h (IP with anti-KCC2 antibodies) before centrifugation (13,000×g at 4° C. for 15-20 min).

Samples were subjected to electrophoresis on polyacrylamide gradient gels (4-12% bis-tris gels) and transferred to nitrocellulose membranes. Membranes were rinsed with TBS-tween solution (TBST; Invitrogen) and blocked with 5% (w/v) skim milk. The membranes were then immunoblotted in 5% (w/v) skim milk in TBST with a primary antibody against KCC2 (rabbit, 07-432; Millipore; 1:1000), or phosphotyrosine (mouse, clone 4G10, Millipore, 1:1000), and a mouse primary antibody against neuron-specific beta-III tubulin overnight at 4° C. (clone 2G10; T8578; Sigma-Aldrich; 1:1000). They were then washed three times with distillated water, three times with TBST-milk solution and incubated for one hour at RT with goat secondary antibodies anti-rabbit IRDye 800 (1:3000; LI-COR), goat anti-mouse IRDye 700 (1:3000; LI-COR) or donkey anti-sheep DyLight 800 (1:3000; Rockland). Membranes were then washed again with TBST three times and one time with TBS. Fluorescence was detected and the relative intensities of immunoblot bands were determined using Odyssey infrared imaging system (LI-COR Bioscience).

For immunoblots without immunoprecipitation, the following antibodies were used for immunodetection of total KCC2, KCC2 phospho S940, total SPAK/OSR1, SPAK phospho Ser373/OSR1 phospho S325: rabbit anti-KCC2 (07-432; Millipore; 1:1000), rabbit anti-KCC2 pS940 (p1551-940, phosphosolutions), sheep anti-SPAK (S551D, third bleed, Dundee) and sheep anti-phospho-SPAK (Ser373)/phospho-OSR1 (Ser325) antibodies (S670B, second bleed, Dundee).

### Kinase Profiling Assays

Kinase activity profiles (data not shown) were performed using radiometric assays to measure kinase catalytic activity for PCPZ concentrations ranging 0.1-500 μM in DMSO or CLP257 concentrations ranging 0.1-1000 μM in DMSO, in the presence of 70 μM ATP. Equimolar DMSO concentrations were used as control. Additional information on the kinase profiler assay can be obtained from the contractor's website. (https://www.eurofinsdiscoveryservices.com/cms/cms-content/services/in-vitro-assays/kinases/kinase-profiler).

### Human Tissue and Multi-Electrode Recordings

Patients or their legal guardians gave informed consent to participate in the study. Patients (13 patients, 3 males, 4 female, age range: 16-54 years) diagnosed with mesial temporal lobe epilepsy with hippocampal sclerosis underwent multimodal preoperative neurological, neurophyschological and psychiatric evaluations as well as video-electroencephalographic recording, magnetic resonance imaging and fluorodeooxyglugose positron emission tomography. Cortectomies were performed at Sainte-Anne Hospital by neurosurgeons B. Devaux and B. Turak and at Pitié-Salpêtriére Hospital by neurosurgeons S. Clemenceau and B. Mathon. Cortical specimens collected in the operating room were immediately transported in ice-cold (0-4° C.), oxygenated solution (O₂/CO₂ 95/5%), containing (in mM): N-methyl-d-glucamine 93, KCl 2.5, NaH₂PO₄ 1.2, NaHCO₃ 30, HEPES 20, D-glucose 20, ascorbic acid 5, sodium pyruvate 3, MgSO₄ 10 and CaCl₂ 0.5 (300-310 mOsm, pH7.4) and transported within 15 minutes to the laboratory. Transverse hippocampal-subicular slices (400 m thick) were prepared in the same solution using a vibratome (HM650V, Microm). They were maintained for 20-30 minutes at 37° C. and then at room temperature in an interface chamber containing artificial cerebrospinal fluid (ACSF) composed of (in mM): D-glucose 10, KCl 3.5, NaHOC₃ 26, NaH₂PO₄ 1.25, NaCl 126, CaCl₂ 1.6 and MgCl₂ 1.2 (290 mOsm), equilibrated with 5% CO₂ in 95% 02.

Multielectrode array recordings were performed using a MEA2100 station (Multi Channel Systems) equipped with a 120-microelectrode array chamber (custom 12×10 layout, 30 μm TiN electrodes spaced 1 mm vertical and 1.5 mm horizontal). Slices were maintained in the recording chamber using a home-made platinum-nylon harp and superfused with pre-warmed (37° C.) oxygenated ACSF at a rate of 6 ml/min. Slices were imaged using a video-microscope table (MEA-VMT1, Multi Channel Systems) in order to register the location of electrodes with respect to the slice. Extracellular signals were acquired at a sampling rate of 10 kHz using Multi Channel Experimenter (Multi Channel Systems) and analyzed offline using homemade software (Matlab, The Mathworks).

### Mouse Model of Temporal Lobe Epilepsy and ECoG Recordings

Our procedures were all approved by local ethics committee (Charles Darwin, Apafis 202008050923999 v4). 8 weeks-old male C57B16/J mice were used in a pilocarpine-based model of temporal lobe epilepsy as described [27]. In brief, animals received an intraperitoneal (ip) injection of LiCl (423 mg/kg) and scopolamine (1 mg/kg), respectively 18-24 hours and 1 hour prior to pilocarpine. Pilocarpine (75 mg/kg) was then injected ip and mice were continuously monitored for the onset of status epilepticus (SE). In mice in which the first pilocarpine injection had not triggered SE after one hour, a second injection at 20 mg/kg was performed. SE was interrupted one hour after onset by ip injection of diazepam and ketamine (both at 10 mg/kg). All animals received subcutaneous injection of 500 μl of NaCl 0.9% 1 hour and 4 hours after SE termination in order to minimize dehydration. They were monitored daily for weight, behavior and were provided with enriched liquid food (Fortimel Energy®) for the first 5-7 days post SE. Mice showing >25% weight loss for over 3 consecutive days were killed and discarded from further analysis.

For chronic PCPZ treatment, control and epileptic mice received two daily ip injections of either NaCl 0.9% or PCPZ (2 mg/kg in NaCl 0.9%), 10-12 hours apart (typically 9 AM and 8 PM).

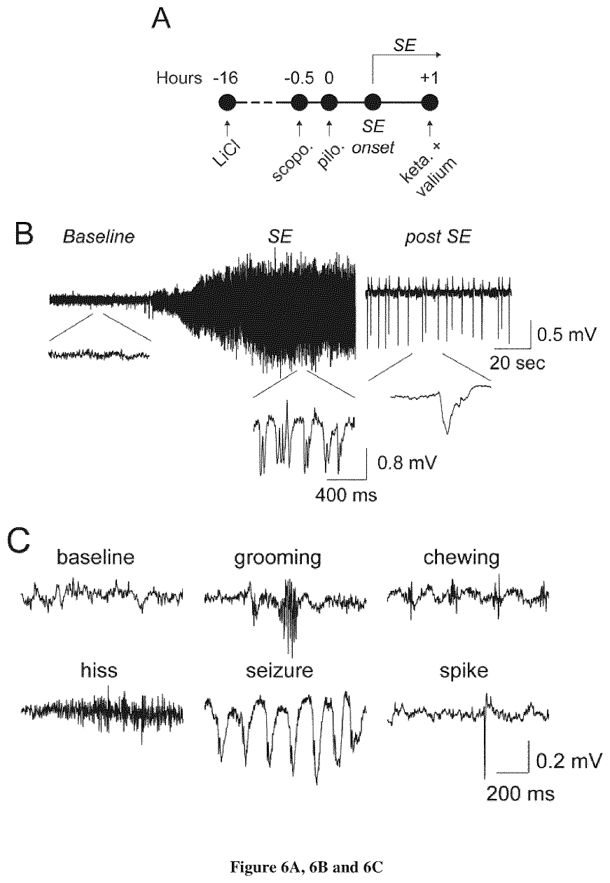

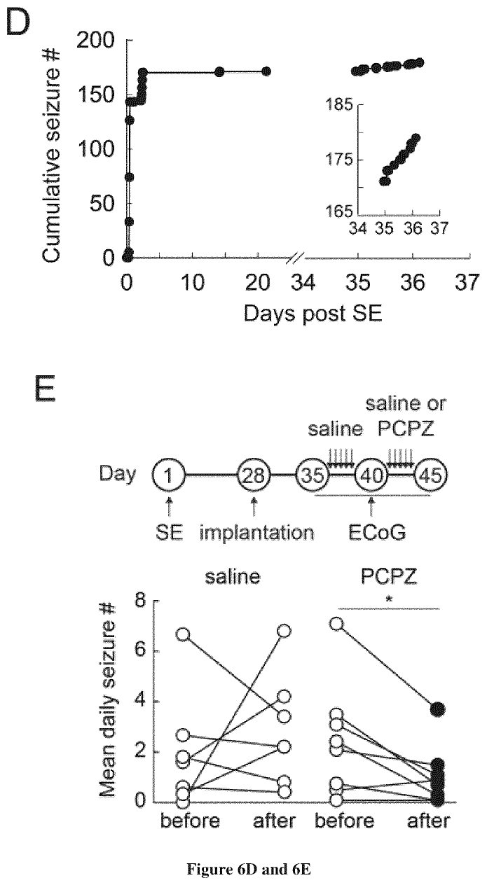

ECoG probe implantation was performed 28 days post SE in all mice except for 1 mouse that was implanted prior to SE induction in order to monitor chronic seizure onset (FIG. 6D). Mice were anesthetized with 4.5% isoflurane and maintained with 2-2.5%. Telemetric ECoG probes (model A3028B, OpenSourceInstruments) were implanted subcutaneously and connected to stainless steel screws (1 mm diameter) stereotaxically inserted in the skull in holes drilled above the right hippocampus (1.94 mm posterior and 1.25 mm lateral with respect to Bregma) and above the cerebellum for reference. Screws were attached to the skull with dental cement. Mice received sc buprenorphine injection (0.05 mg/kg) before and after surgery in order to minimize pain. One week after implantation, ECoG signal (0.3-160 Hz) were recorded continuously using a telemetric Octal Data Receiver (OpenSourceInstruments), amplified 100× and acquired at 512 Hz. Recordings were analyzed offline using Event Classification Processor ECP20 under NeuroArchiver 132. Seizures were detected and distinguished from other ECoG events (spikes, grooming, hiss . . . ) semi-automatically, based on a preset signal library and verified manually as ECoG signals including single or complex polyspikes over a period of 15 seconds or more.

### Statistics

Sampling corresponds to the number of cells for electrophysiology and ICC, quantum dots for SPT, cultures or animals for biochemistry, animals for in vivo experiments, and slices and patients for human tissue recordings. Sample size selection for experiments was based on published experiments, pilot studies, as well as in-house expertise. Means are shown±SEM, median values are indicated with their interquartile range (IQR, 25-75%). Means were compared using Student's parametric t test when statistical conditions were verified (normal distribution and homoscedasticity). Otherwise, a non-parametric Mann-Whitney (MW) test was performed.

For paired measurements, Wilcoxon test were used unless statistical conditions were verified for Student's paired t-test. Statistical tests were performed using SigmaPlot 13 (Systat Software). Differences were considered significant for p-values less than 5% (* p<0.05, ** p<0.01, * ** p<0.001). Error bars correspond to SEM.

### Results

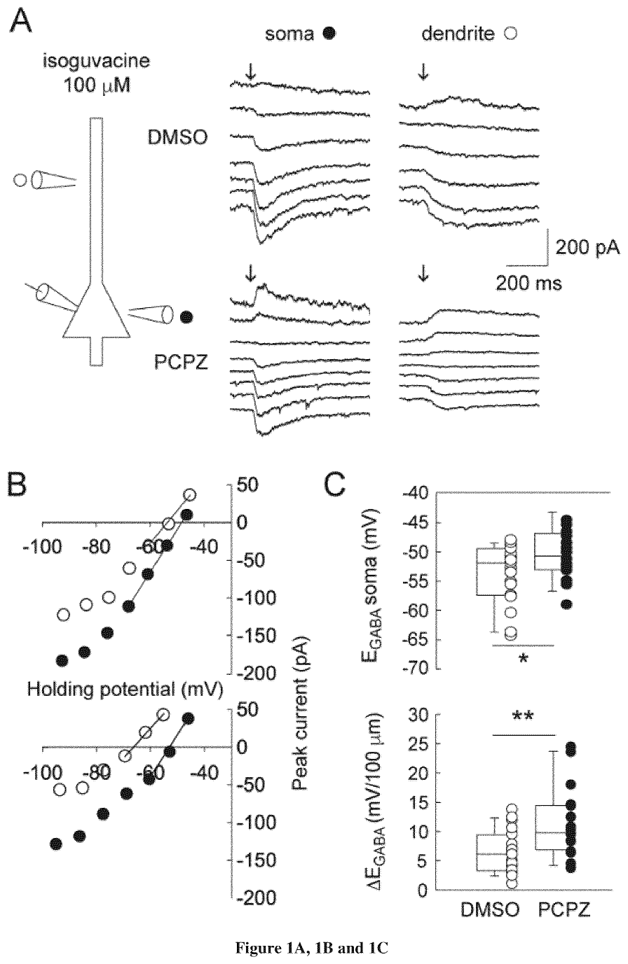

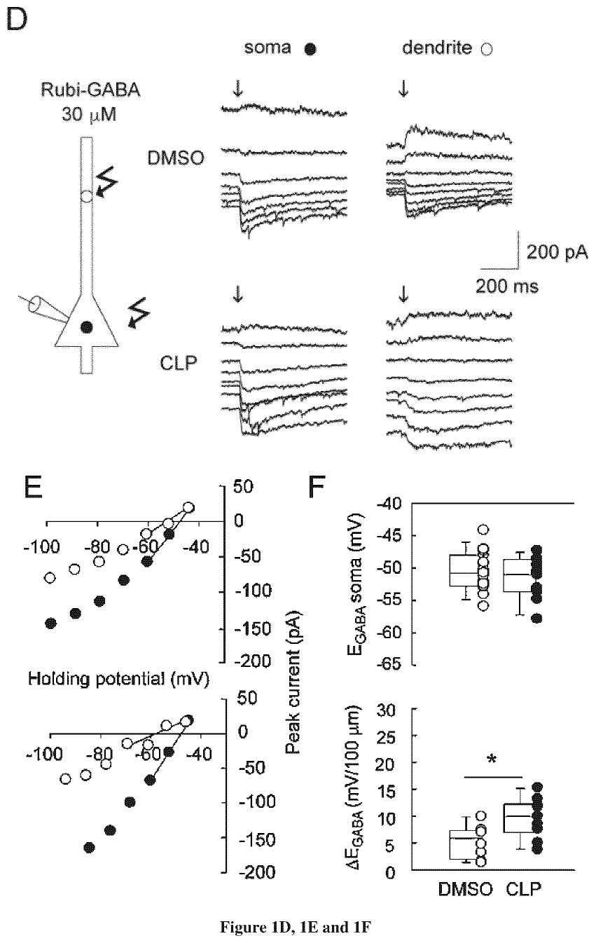

Prochlorperazine (PCPZ), an antipsychotic phenothiazine derivative, has been suggested to enhance KCC2 function in lesioned spinal motoneurons [7]. However, it is not known whether this effect also applies to cortical neurons and what the underlying mechanisms of action might be. Therefore, we first evaluated whether PCPZ potentiates KCC2 in rat hippocampal neurons in vitro and compared its effects to those of another candidate KCC2 enhancer, CLP257 [6]. KCC2 function was assessed by comparing the reversal potential of GABAA receptor-mediated currents (E_{GABA}) evoked by focal application of GABA or the GABAA receptor agonist isoguvacine (see Methods) on the soma vs. dendrites of neurons whole-cell patch-clamped with 29 mM chloride-containing internal solution [14-16] (FIG. 1A-C). A somato-dendritic gradient of E_{GABA} (ΔE_{GABA}) of 6.56±0.83 mV/100 μm was observed in control, untreated neurons. Upon 2-hour application of 10 μM PCPZ, this gradient was significantly increased to 11.50±1.53 mV/100 μm (p=0.006). We also observe a small but significant depolarization of somatic E_{GABA} in PCPZ-treated neurons in comparison with control neurons (−50.17±1.19 vs. −53.64±1.16 mV/100 μm, p=0.046). This enhanced somato-dendritic ΔE_{GABA} reveals enhanced transmembrane chloride extrusion capacity, suggesting increased KCC2 net function upon PCPZ application. Similarly, 2-hour application of CLP257 (100 μM) increased ΔE_{GABA} from 5.32±1.08 to 9.70±1.13 mV/100 μm (p=0.014) (FIG. 1D-F). These data demonstrate both PCPZ and CLP257 act as genuine KCC2 enhancers in rat hippocampal neurons.

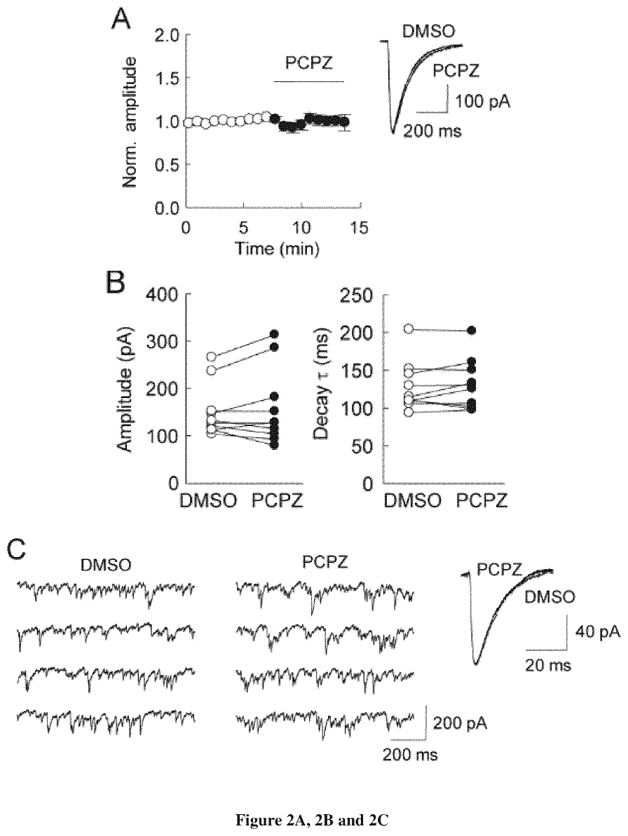

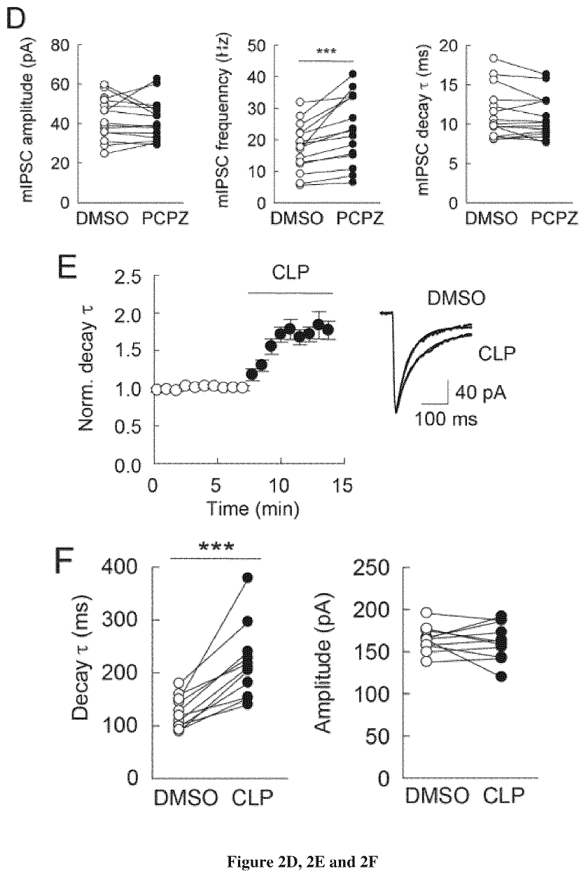

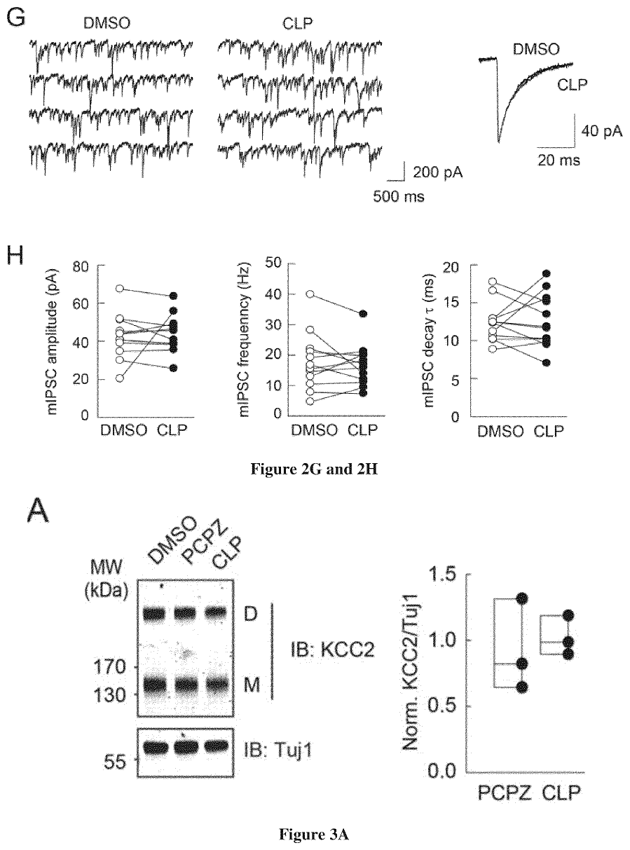

CLP257 has been suggested to act directly to potentiate GABAA receptors [9]. Since GABAAR function itself modulates KCC2 membrane expression and function [17], we asked whether both PCPZ and CLP257 may act primarily to modulate GABAAR function. First, we tested the effect of acute PCPZ or CLP257 application on extrasynaptic GABAAR function, as assessed by focal application of isoguvacine (100 μM) or uncaging of low concentration (30 μM) of Rubi-GABA (FIG. 2). PCPZ had no effect on either the amplitude (157.51±25.46 vs. 150.73±17.50 pA for control, p=0.48) or the decay time constant (130.81±10.48 vs. 127.66±10.23 ms, p=0.362) of GABAA receptor-mediated currents (FIG. 2A-B). Instead, CLP257 was found to increase the decay time constant of GABAA receptor-mediated currents (218.22±21.05 vs. 124.39±9.12 ms, p<0.001) with no detectable effect on their amplitude (162.04±6.62 vs 164.70±4.56 pA for control, p=0.66; FIG. 2E-F). In order to test the effect of CLP257 and PCPZ specifically on synaptic GABAA receptors, miniature inhibitory postsynaptic currents (mIPSCs) were recorded from hippocampal neurons before and upon application of the drugs. Neither PCPZ nor CLP257 had detectable effects on mIPSCs amplitude (42.92±2.55 vs. 42.49±2.54 pA, p=0.797 and 43.77±2.86 vs. 42.32±3.40 pA, p=0.622, respectively) or decay time constant (10.17±0.71 vs. 10.66±0.80 ms, p=0.086 and 12.53±1.01 vs. 12.36±0.75 ms, p=0.86, respectively). However, PCPZ but not CLP257 marginally increased mIPSCs frequency (23.38±2.38 vs. 17.42±1.69 ms, p<0.001 and 16.58±2.00 vs. 17.71±2.74 ms, p=0.51, respectively, FIG. 2C-D, G-H). Together, our results demonstrate that while CLP257 potentiates both KCC2 and extrasynaptic GABAA receptors in primary hippocampal neurons, PCPZ enhances KCC2 function without acting directly on GABAA receptors.

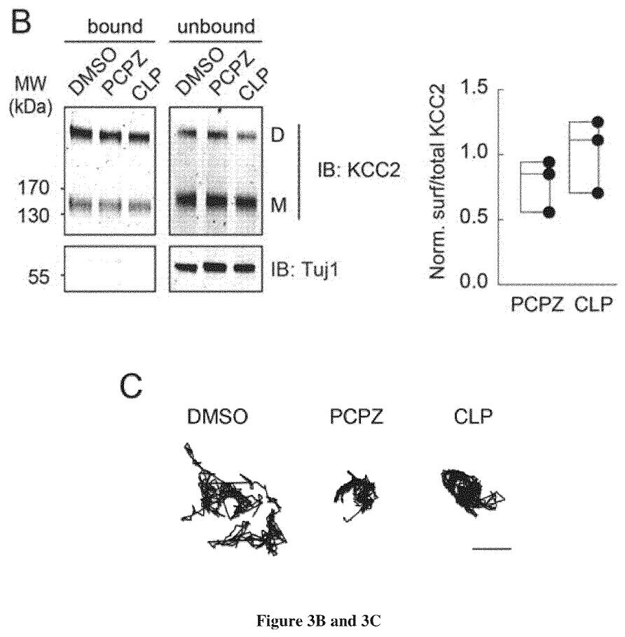

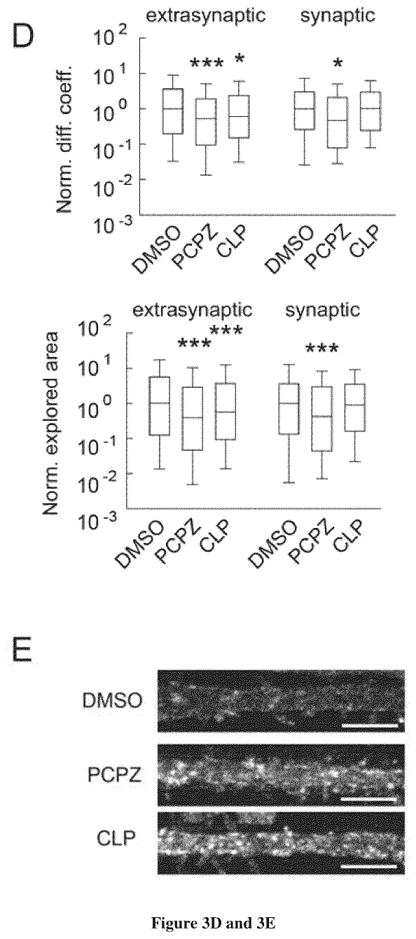

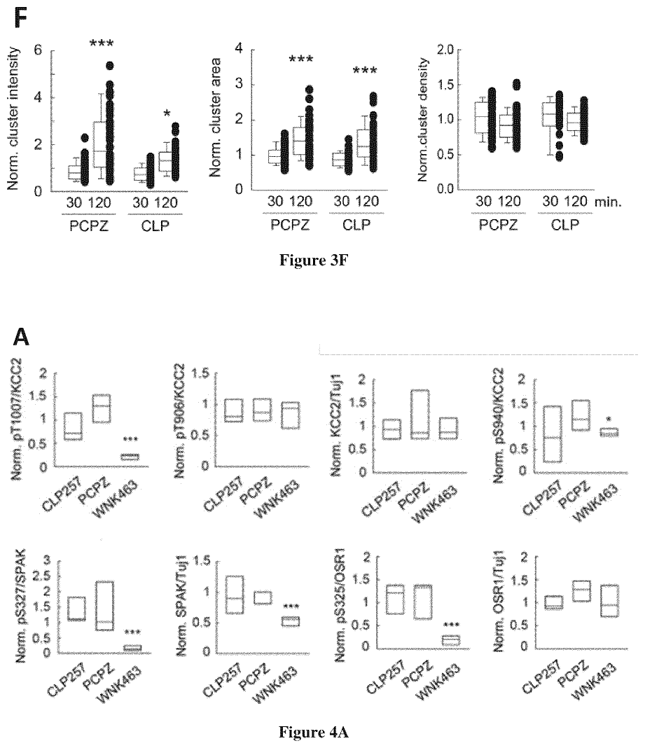

We next investigated the mechanisms of potentiation of KCC2 function by PCPZ and CLP257. Enhanced net KCC2 function may result from either increased intrinsic enzymatic activity or increased membrane expression of the transporter, or both. We therefore asked whether increased KCC2 function upon PCPZ or CLP257 application was associated with increased surface expression of the transporter using surface biotinylation assay. In these experiments, we observed that 2-hour application of either PCPZ or CLP257 had no effect on either the total (FIG. 3A, p=0.137 and p=0.893, respectively) or the ratio of surface to total KCC2 expression (FIG. 3B, p=0.735 and p=0.8, respectively). Therefore, enhanced KCC2 function by PCPZ or CLP257 does not reflect increased expression of the transporter.

KCC2 function may also be regulated by fine-tuning of its diffusion and clustering properties [18]. Thus, increased KCC2 function is often associated with reduced membrane diffusion and increased clustering [17]. We therefore tested the effect of PCPZ and CLP257 on these properties in hippocampal neurons. Membrane lateral diffusion of recombinant Flag-tagged KCC2 was reduced both near inhibitory synapses and in extrasynaptic domains upon 2-hour application of PCPZ or CLP257. This effect was detected in quantum dot-based single particle tracking experiments by a reduced diffusion coefficient (extrasynaptic: −36.54±14.91% p<0.001; synaptic: −26.22±14.00% p=0.011 for PCPZ and extrasynaptic: −20.74±4.85% p=0.003; synaptic: +9.39±11.21% p=0.97 for CLP257) and explored area (extrasynaptic: −39.70±17.18% p<0.001; synaptic: −30.632±25.44% p<0.001 for PCPZ and extrasynaptic: −25.14±2.73% p<0.001; synaptic: +4.93±19.40% p=0.80 for CLP257; FIG. 3C-D). It was associated with an increased clustering of the transporter, as detected in immunocytochemistry assays revealing enhanced cluster intensity (+109±61.44% p<0.001 and +33.94±13.64% p=0.005, respectively) and area (+45.25±18.07% p<0.001 and +36.30±20.40% p<0.001, respectively) with no significant change in cluster density (−6.58±8.37% p=0.13 and −3.48±4.20% p=0.34; FIG. 3E-F). Importantly, this effect was observed after 2 hours of treatment with PCPZ or CLP257 but not after 30 minutes only (FIG. 3F). Altogether, our results suggest both PCPZ and CLP257 act to enhance KCC2 net function and promote KCC2 clustering, with no effect on total KCC2 expression or surface/total expression ratio. These results indicate PCPZ and CLP257 may induce a redistribution of membrane-inserted KCC2 resulting in enhanced clustering.

KCC2 membrane stability, clustering and turnover are under control of a variety of post-translational mechanisms, including phosphorylation of key residues—in particular in its large carboxy-terminal domain—that regulate its net transport function [18-21]. Phosphorylation of serine 940 (S940) by protein kinase C is known to increase the membrane stability of KCC2 and its function ([50, 52]). In contrast, phosphorylation of 906 and 1007 threonine residues (T906, T1007) by the With-no-lysine (K) (WNK) kinase pathway has been shown to negatively regulate KCC2 function in cortical neurons by affecting its membrane diffusion and clustering [17,22]. We therefore asked whether PCPZ and CLP257 could alter the phosphorylation level of these specific residues to promote KCC2 function.

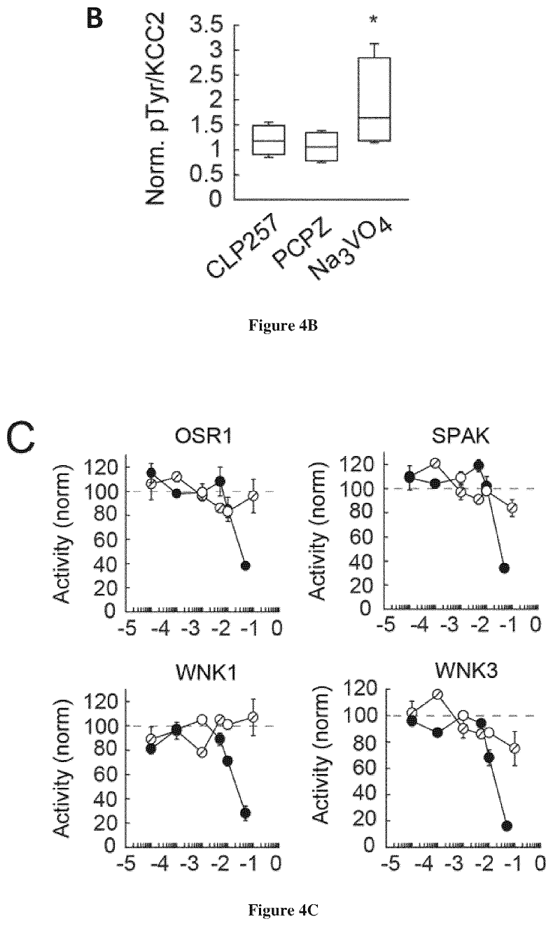

Immunoprecipitation assays from adult rat hippocampo-cortical slices (FIG. 4A, see Methods) revealed that CLP257 and PCPZ did not significantly affect KCC2 phosphorylation at either S940 (−19.70±34.40% p=0.597 and +20.30±18.70% p=0.339), T1007 (−18.50±17.10% p=0.340 and +26.40%±17.00% p=0.195 respectively) or T906 (−12.70±10.90% p=0.308 and −10.20±10.40% p=0.384 respectively) residues. Total KCC2 expression was also unaffected (−6.92±11.80% p=0.590 and +12.1±32.8% p=0.731 respectively). In agreement with our results on pT906 and pT1007, CLP257 and PCPZ did not significantly affect phosphorylation of the WNK effectors SPAK (S373 residue, +33.30±24.00% p=0.238 and +36.90±48.40% p=0.488 respectively) and OSR1 (S325 residue, +11.50±18.50% p=0.568 and +11.80±23.50% p=0.641 respectively) or the total expression of SPAK (−5.97±17.4% p=0.748 and −12.30±6.3 5% p=0.124 respectively) or OSR1 (−2.24±8.27% p=0.800 and +26.3±12.8% p=0.108 respectively). In contrast, as expected, 2-hour application of the pan-WNK inhibitor WNK463 [51], reduced T1007 (−78.702±3.27% p<0.001) and, to a lesser extent, S940 (−14.40±4.67% p=0.0365) phosphorylation, with no effect on T906 phosphorylation (−13.70±12.5% p=0.333) or total KCC2 expression (−7.64±13.00% p=0.588). These effects of WNK463 were associated with a significant reduction of SPAK (S327, −84.10±4.57% p<0.001) and OSR1 (S325, −81.00±5.56% p<0.001) phosphorylation. Consistent with the above results, PCPZ and CLP257 showed no direct inhibitory effect on either WNK1, WNK3, SPAK or OSR1 in in vitro kinase assays (FIG. 4C). These results suggest that CLP257 and PCPZ potentiate KCC2 independent of PKC- or WNK/SPAK-mediated phosphorylation.

Finally, phosphorylation of Y903 and Y1087 KCC2 residues was reported to alter KCC2 clustering and function, both in heterologous cells and in hippocampal neurons [21, 49]. We therefore tested the effect of CLP257 and PCPZ on the phosphorylation of these residues in adult rat hippocampo-cortical slices (FIG. 4B). Tyrosine phosphatase inhibitor sodium orthovanadate (Na₃VO₄) but neither CLP257 nor PCPZ significantly increase tyrosine phosphorylation of KCC2 (+89.10±45.40% p=0.029 and +19.00±15.00% p=0.343 and +6.38±14.7% p=1 respectively), suggesting that CLP257 and PCPZ do not act on the pathways regulating tyrosine phosphorylation of KCC2.

Together our results demonstrate that both PCPZ and CLP257 potentiate KCC2 function, reduce its diffusion and increase its clustering via a mechanism/pathway that does not involve phosphorylation of canonical residues.

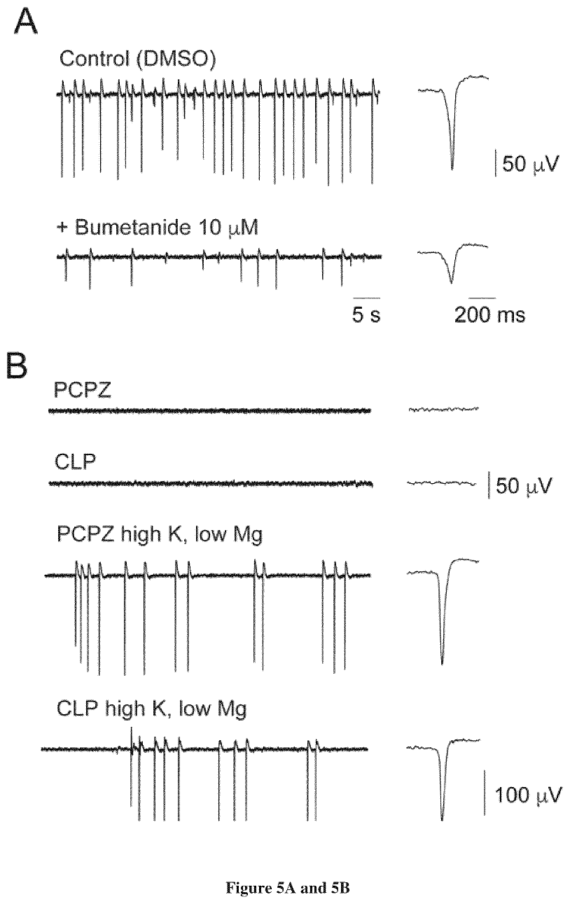

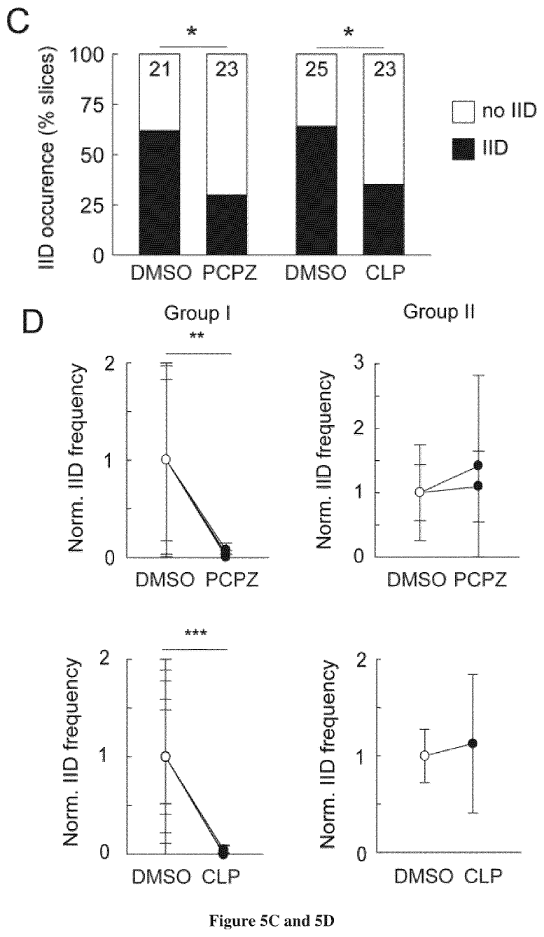

Several arguments support that promoting KCC2 function may be beneficial in preventing epileptiform activity. Thus, subicular neurons with reduced KCC2 expression are paradoxically excited during interictal-like events recorded in vitro in postoperative tissue from mesial temporal lobe epilepsy (mTLE) patients [24]. In addition, KCC2 overexpression prevented epileptiform activity induced by artificial recruitment of somatic inhibition in a mouse model of acute seizures [25]. We first tested the effect of PCPZ and CLP257 on tissue samples resected from patients with mesial temporal lobe epilepsy associated with hippocampal sclerosis and recorded using multielectrode arrays. As reported earlier [24,26], 29 out of 46 slices prepared from 13 postoperative samples displayed spontaneous interictal-like activity. Interictal-like discharges (IIDs) had a mean amplitude of 57.33±13.52 μV and occurred at a mean frequency of 0.44±0.10 Hz (FIG. 5A). In 3 of 4 slices, the NKCC1 antagonist bumetanide (10 μM) reduced the amplitude or abolished IIDs, as previously described [24]. We then tested the effect of 2-hour incubation in 10 μM PCPZ or 100 μM CLP257 vs. vehicle only (DMSO; FIG. 5B-D). Overall, both PCPZ and CLP257 significantly reduced the proportion of slices displaying IIDs (p<0.05 and p<0.05 respectively; FIG. 5B-C). In slices from a group of 5 mTLE patients (group I), PCPZ reduced IID frequency by 97.55±13.8% (p<0.001) with no significant change on their amplitude (+9.0% 26.6%, p=0.768). This reduced interictal-like activity was likely not due to cell loss or compromised neuronal integrity as it could be restored by increasing neuronal activity with a high-potassium/low magnesium ACSF (FIG. 5B). In slices from the remaining 2 patients (group II), however, PCPZ failed to reduce either IID frequency (+25.5±15.9%, p=0.356) or amplitude (+18.2±4.8%, p=0.165). Similarly, incubation with CLP257 reduced IID frequency by 96.5±2.31% (p<0.001) with no significant change in their amplitude (+134.4% 127.3%, p=0.402) in slices from 5 out of 6 patients. In slices from the remaining patient however, CLP257 showed little effect on the frequency (+12.6%) and the amplitude (−24.5%) of IIDs (FIG. 5B-D).

Finally, since spontaneous ictal activity is rarely observed in slices from postoperative mTLE tissue [24], we explored the effect of a KCC2 enhancer on seizures occurrence in the lithium-pilocarpine model of mTLE [27] adapted to mice (FIG. 6). Since PCPZ but not CLP 257 [6] may be readily used in vivo, we performed these experiments first with PCPZ. Status epilepticus (SE) was induced by intraperitoneal (i.p.) injection of 75 mg/kg of the muscarinic agonist pilocarpine 16 hours after injection of 423 mg/kg LiCl (see Methods, FIG. 6A). Mice usually developed SE within 10-20 min of pilocarpine injection, as detected by telemetric EEG recordings of cortical activity (FIG. 6B). SE was interrupted after 1 hours by i.p. injection of valium and ketamine (10 mg/kg each). ECoG recordings displayed intense IID activity and occasional seizures for hours to a few days post-SE (FIG. 6D) followed by a seizure-free, latent period lasting 3-4 weeks. From days 35 post-SE, regular, spontaneous seizures were distinguished from physiological events using a library-based detection software (FIG. 6C) and were detected daily with an average of 1.87±0.47 seizure per day (n=19 mice). We tested the effect of two daily PCPZ injections (2 mg/kg, i.p., 11 hours apart) during the chronic phase (days 40-45), on the occurrence of spontaneous seizures. PCPZ injections led to a 3-fold reduction in seizure frequency as compared to control group receiving saline injections (0.95±0.42 vs. 2.86±0.83 per day, p=0.024; FIG. 6E). We conclude that PCPZ shows antiepileptic properties and reduces both interictal-like discharges in postoperative tissue from mTLE patients and chronic seizures in an animal model of mTLE.

### Discussion

Our data provide the first proof-of-concept of the therapeutic potential of a KCC2 enhancer in a form of drug-resistant epilepsy and suggest that PCPZ may have therapeutic potential in this and related indications.

Several forms of epilepsy are associated with KCC2 downregulation [28], leading to paradoxically excitatory signaling by GABAA receptors [24,29]. Although pro-GABAergic drugs such as benzodiazepines and other GABAA receptor-positive allosteric modulators are often effective in epilepsy [30], acting to directly potentiate GABA signaling when intraneuronal chloride levels are abnormal may exacerbate rather than prevent seizures. Restoring neuronal chloride homeostasis therefore appears as a promising therapeutic strategy in forms of pharmaco-resistant epilepsy. One approach to reduce intraneuronal chloride consists in blocking chloride import, using NKCC1 antagonists such as bumetanide. Although this approach was used successfully in some animal models of CNS disorders [31-34] it is not optimal as i) bumetanide is largely bound to plasma proteins and very poorly crosses the blood brain barrier [35] and ii) NKCC1 is widely expressed in many peripheral organs and tissues, raising issues regarding the number of undesirable side effects. In fact, two clinical trials (NCT01434225; NCT00830531) were conducted to evaluate the potential of bumetanide for the treatment of neonatal seizures. One of them, the NEMO trial [36], was interrupted prematurely as bumetanide failed to reach the primary endpoint and because hearing loss was observed in 3/11 surviving patients, likely due to the involvement of NKCC1 in the maturation of cochlear hair cells [37].

Instead, KCC2 is almost exclusively expressed in neurons, making it a prime target for neurological disorders associated with deficits in neuronal chloride homeostasis such as epilepsy. Acing to promote KCC2 function, even when its expression may be reduced in the pathology, may help rescue neuronal chloride export and inhibitory GABA signaling. CL-058 was the first compound identified as a candidate KCC2 enhancer by screening of small molecule libraries on a mouse neuroblastoma cell line [6]. It was then chemically optimized into CLP-257 and CLP-290 with improved EC₅₀ and half-life for in vivo applications. Whereas CLP drugs were initially described as specific KCC2 enhancers with little or no activity on other cation-chloride cotransporters [6], a more recent study challenged this view and showed no effect on KCC2 function [9]. Instead, this study revealed a direct binding and potentiating action on GABAA receptors. Although still controversial [10], these results challenged the view of CLP drugs as pure KCC2 enhancing molecules. Consistent with these observations, we now report that CLP257 acts both as a KCC2 enhancer and a positive, allosteric modulator of extrasynaptic but not synaptic GABAA receptors in rat hippocampal neurons. These data may help reconcile apparently conflicting results obtained on recombinant GABAA receptors expressed in heterologous cells [9-10]. CLP-290 induced partial functional recovery in mouse models of chronic pain upon peripheral nerve injury [6] and paralysis upon spinal cord injury [38], both associated with altered KCC2 expression and GABA signaling. However, no study so far explored its effects in the context of epilepsy. Only one study reported the effect of CLP-257 on epileptiform activity pharmacologically induced in vitro, with somewhat contrasting results [39]. How these effects relate to spontaneous ictogenesis in vivo remained unknown. Our new data now demonstrate that CLP257 increased KCC2 function through enhanced membrane clustering of the transporter and strongly reduces spontaneous epileptiform activity recorded in vitro from human postoperative tissue from mTLE patients.

PCPZ is commonly known as a dopamine D2 antagonist, although it also interacts with 5-HT3 and nicotinic acetylcholine receptors. It was first suggested as a candidate KCC2 enhancer by screening of the Prestwick library on HEK cells expressing recombinant KCC2 [7]. It was then shown to reduce the depolarizing driving force of evoked IPSCs in immature (P6) rat spinal motoneurons upon spinal cord injury. This effect was associated with an apparent increase in KCC2 surface expression in these cells. However, whether PCPZ acts similarly in CNS neurons was unknown, as were the underlying mode of action. We now demonstrated that PCPZ acts as a genuine KCC2 enhancer in rat hippocampal neurons. Unlike CLP257, PCPZ showed no direct functional effect on GABAA receptors. Functional enhancement of KCC2 in neurons is associated with increased clustering of the transporter with no apparent change in total or surface expression, in contrast to earlier observations in heterologous cells [7]. Surprisingly this effect does not involve changes in the phosphorylation of canonical serine, threonine or tyrosine residues, suggesting it may instead involve changes in protein-protein interactions. For example, interaction of KCC2 with the SNARE proteins SNAP23 and syntaxin 1A is essential for the mZnR/GPR39-dependent upregulation of KCC2 activity [45]. However, this modulation involves regulation of KCC2 membrane insertion, which was not observed in our surface biotinylation assays after a treatment with CLP257 or PCPZ. Others candidates such as CCC CIP1 [46], Neto2 [47], CKB [48] and gephyrin [44] are of particular interest since they interact with KCC2 and increase its function. Finally, KCC2 enhancers may act to promote KCC2 accumulation into lipid rafts, resulting in enhanced transporter function [21]. PCPZ is a first-generation antipsychotic with FDA approval and indications in schizophrenia, migraine as well as nausea and vomiting. PCPZ administration is primarily intramuscular and oral for psychiatric indications, although parenteral and rectal administration is also possible [40]. Side effects are generally mild in adults [41] but adverse effects such as sedation and extrapyramidal symptoms have been reported in children [43]. In some rare cases, PCPZ administration in children was reported to cause seizures [42]. Instead, we now provide evidence that PCPZ shows significant antiepileptic properties in two experimental models. First, like CLP257, PCPZ significantly suppressed spontaneous, interictal-like discharges recorded in vitro from slices of postoperative brain samples of 5 out of 7 mTLE patients. This effect is comparable to that of bumetanide on various postoperative epileptic brain samples [24, 29, 43], adding to the evidence that rescuing neuronal chloride transport may be an effective anti-epileptic strategy. Second, it reduced spontaneous seizure occurrence by 3-fold in a mouse model of mTLE. These results demonstrate that PCPZ may act as an effective antiepileptic drug, alone or in combination with pro-GABAergic compounds. It will now be interesting to test its effects on seizure occurrence in patients with pharmaco-resistant epilepsies.

### REFERENCES

Throughout this application, various references describe the state of the art to which this invention pertains. The disclosures of these references are hereby incorporated by reference into the present disclosure.
1. GBD 2016 Epilepsy Collaborators. Global, regional, and national burden of epilepsy, 1990-2016: a systematic analysis for the Global Burden of Disease Study 2016. Lancet Neurology 18(4), 357-375 (2019).
2. Mesraoua, B., et al. Novel therapies for epilepsy in the pipeline. Epilepsy Behav 97, 282-2903 (2019).
3. Blaesse, P., et al. Cation-chloride cotransporters and neuronal function. Neuron 61(6):820-38 (2009).
4. Huberfeld, G., et al. Hippocampus and epilepsy: Findings from human tissues. Rev Neurol (Paris) 171(3):236-51 (2015).
5. Pressler, R. M., et al. Bumetanide for the treatment of seizures in newborn babies with hypoxic ischaemic encephalopathy (NEMO): an open-label, dose finding, and feasibility phase ½ trial. Lancet Neurology 14, 469-477 (2015).
6. Gagnon, M., et al. Chloride extrusion enhancers as novel therapeutics for neurological diseases. Nature Medicine 19(11):1524-8 (2013).
7. Liabeuf, S., et al. Prochlorperazine Increases KCC2 Function and Reduces Spasticity after Spinal Cord Injury. J Neurotrauma 34(24):3397-3406 (2017).
8. Tang, X., et al. Pharmacological enhancement of KCC2 gene expression exerts therapeutic effects on human Rett syndrome neurons and Mecp2 mutant mice. Science Transl Medicine 11(503) eaau0164 (2019)
9. Cardarelli, R. A., et al. The small molecule CLP257 does not modify activity of the K+-Cl— co-transporter KCC2 but does potentiate GABA A receptor activity. Nature Medicine 23(12):1394-1396 (2017).
10. Gagnon, M., et al. Reply to The small molecule CLP257 does not modify activity of the K+-Cl— co-transporter KCC2 but does potentiate GABA A receptor activity. Nature Medicine 23(12):1396-1398 (2017)
11. Vossel, K. A., et al. Incidence and impact of subclinical epileptiform activity in Alzheimer's disease. Ann Neurol. 80(6):858-870 (2016)
12. Gent, T. C, et al. Epileptiform activity during inert gas euthanasia of mice. PLoS One. 13(4):e0195872 (2018)
13. Bentes, C., et al. Frequency of post-stroke electroencephalographic epileptiform activity—a systematic review and meta-analysis of observational studies. Eur Stroke J. 2(4):361-368 (2017).
14. Gauvain, G., et al. The neuronal K—Cl cotransporter KCC2 influences postsynaptic AMPA receptor content and lateral diffusion in dendritic spines. Proceedings of the National Academy of Sciences of the United States of America 108, 15474-15479 (2011).
15. Khirug, S., et al. GABAergic depolarization of the axon initial segment in cortical principal neurons is caused by the Na—K-2Cl cotransporter NKCC1. The Journal of neuroscience: the official journal of the Society for Neuroscience 28, 4635-4639 (2008).
16. Otsu, Y., et al, Cation-chloride cotransporters and the polarity of GABA signalling in mouse hippocampal parvalbumin interneurons. The Journal of physiology 598, 1865-1880 (2020).
17. Heubl, M., et al. GABAA receptor dependent synaptic inhibition rapidly tunes KCC2 activity via the Cl—-sensitive WNK1 kinase. Nature communications 8, 1776 (2017).
18. Côme, E., et al. Reciprocal Regulation of KCC2 Trafficking and Synaptic Activity. Frontiers in cellular neuroscience 13(2019).
19. Kahle, K. T., et al. K—Cl cotransporters, cell volume homeostasis, and neurological disease. Trends in molecular medicine 21, 513-523 (2015).
20. Lee, H. H., et al. Tyrosine phosphorylation regulates the membrane trafficking of the potassium chloride co-transporter KCC2. Molecular and cellular neurosciences 45, 173-179 (2010).
21. Watanabe, M., et al. Clustering of neuronal K+-Cl— cotransporters in lipid rafts by tyrosine phosphorylation. The Journal of biological chemistry 284, 27980-27988 (2009).
22. de Los Heros, P., et al. The WNK-regulated SPAK/OSR1 kinases directly phosphorylate and inhibit the K+-Cl— co-transporters. The Biochemical journal 458, 559-573 (2014).
23. Friedel, P., et al. WNK1-regulated inhibitory phosphorylation of the KCC2 cotransporter maintains the depolarizing action of GABA in immature neurons. Sci Signal 8, ra65 (2015).
24. Huberfeld, G., et al. Perturbed chloride homeostasis and GABAergic signaling in human temporal lobe epilepsy. The Journal of neuroscience: the official journal of the Society for Neuroscience 27, 9866-9873 (2007).
25. Magloire, V., et al. KCC2 overexpression prevents the paradoxical seizure-promoting action of somatic inhibition. Nature communications 10, 1225 (2019).
26. Cohen, I., et al. On the origin of interictal activity in human temporal lobe epilepsy in vitro. Science 298, 1418-1421 (2002).
27. Foffani, G., et al. Reduced spike-timing reliability correlates with the emergence of fast ripples in the rat epileptic hippocampus. Neuron 55, 930-941 (2007).
28. Kaila, K., et al. Cation-chloride cotransporters in neuronal development, plasticity and disease. Nature reviews. Neuroscience 15, 637-654 (2014).
29. Pallud, J., et al. Cortical GABAergic excitation contributes to epileptic activities around human glioma. Science translational medicine 6, 244ra289 (2014).
30. Greenfield, L. J., Jr. Molecular mechanisms of antiseizure drug activity at GABAA receptors. Seizure 22, 589-600 (2013).
31. Banerjee, A., et al. Jointly reduced inhibition and excitation underlies circuit-wide changes in cortical processing in Rett syndrome. Proceedings of the National Academy of Sciences of the United States of America (2016).
32. Kourdougli, N., et al. Depolarizing GABA contributes to glutamatergic network rewiring in epilepsy. Annals of neurology 81, 251-265 (2017).
33. Dargaei, Z., et al. Restoring GABAergic inhibition rescues memory deficits in a Huntington's disease mouse model. Proceedings of the National Academy of Sciences of the United States of America 115, E1618-e1626 (2018).
34. Lozovaya, N., et al. Early alterations in a mouse model of Rett syndrome: the GABA developmental shift is abolished at birth. Scientific reports 9, 9276 (2019).
35. Puskarjov, M., et al. Pharmacotherapeutic targeting of cation-chloride cotransporters in neonatal seizures. Epilepsia 55, 806-818 (2014).
36. Pressler, R. M., et al. Bumetanide for the treatment of seizures in newborn babies with hypoxic ischaemic encephalopathy (NEMO): an open-label, dose finding, and feasibility phase ½ trial. The Lancet Neurology 14, 469-477 (2015).
37. Pace, A. J., et al. Ultrastructure of the inner ear of NKCC1-deficient mice. Hear Res 156, 17-30 (2001).
38. Chen, B., et al. Reactivation of Dormant Relay Pathways in Injured Spinal Cord by KCC2 Manipulations. Cell 174, 521-535 e513 (2018).
39. Hamidi, S. & Avoli, M. KCC2 function modulates in vitro ictogenesis. Neurobiology of disease 79, 51-58 (2015).
40. Chokhawala, K. & Stevens, L. Antipsychotic Medications. in StatPearls (StatPearls Publishing Copyright © 2021, StatPearls Publishing LLC., Treasure Island (FL), (2021).
41. Din, L. & Preuss, C. V. Prochlorperazine. in StatPearls (StatPearls Publishing Copyright © 2021, StatPearls Publishing LLC., Treasure Island (FL), 2021).
42. Lin, L. M., et al. The Safety of Prochlorperazine in Children: A Systematic Review and Meta-Analysis. Drug Saf 39, 509-516 (2016).
43. Blauwblomme, T., et al. Gamma-aminobutyric acidergic transmission underlies interictal epileptogenicity in pediatric focal cortical dysplasia. Annals of neurology 85, 204-217 (2019).
44. Al Awabdh, S., et al. Gephyrin Interacts with the K—Cl Cotransporter KCC2 to Regulate Its Surface Expression and Function in Cortical Neurons. J Neurosci. 42, 166-182 (2022)
45. Asraf, H., et al. SNAP23 regulates KCC2 membrane insertion and activity following mZnR/GPR39 activation in hippocampal neurons. iScience 25:103751 (2022)
46. Wenz, M., et al. CIP1 is an activator of the K+-Cl— cotransporter KCC2. Biochem Biophys Res Commun. 381: 388-92 (2009)
47. Ivakine, E. A, et al. Neto2 is a KCC2 interacting protein required for neuronal Cl-regulation in hippocampal neurons. Proc Natl Acad Sci USA 110: 3561-6 (2013)
48. Inoue, K., et al. Brain-type creatine kinase activates neuron-specific K+-Cl— co-transporter KCC2. J Neurochem. 96: 598-608 (2006)
49. Lee, H. H., et al. Tyrosine phosphorylation regulates the membrane trafficking of the potassium chloride co-transporter KCC2. Mol Cell Neurosci. 45: 173-9 (2010)
50. Chamma, I., et al. Activity-dependent regulation of the K/Cl transporter KCC2 membrane diffusion, clustering, and function in hippocampal neurons. J Neurosci. 33: 15488-503 (2013)
51. Yamada, K., et al. Small-molecule WNK inhibition regulates cardiovascular and renal function. Nat Chem Biol. 12: 896-898 (2016)
52. Lee, H. H., et al. Direct protein kinase C-dependent phosphorylation regulates the cell surface stability and activity of the potassium chloride cotransporter KCC2. J Biol Chem 282: 29777-84 (2007)

1. A method for treating refractory epilepsy in a subject in need thereof comprising administering to the subject an effective amount of a KCC2 activator.

2. The method according to claim 1, wherein the KCC2 activator is prochlorperazine, CLP257, CLP290, a CLP257 derivatives, or a CLP290 derivatives.

3. The method according to claim 1, wherein the refractory epilepsy is a refractory temporal lobe epilepsy.

4. A method for treating epilepsy in a subject in need thereof comprising administering to the subject a therapeutically effective amount of prochlorperazine, CLP257, CLP290, a CLP257 derivatives, or a CLP290 derivatives.

5. The method according to claim 4, wherein the epilepsy is a refractory epilepsy.

6. The method according to claim 5, wherein the refractory epilepsy is a refractory temporal lobe epilepsy.

7. (canceled)

8. (canceled)

9. (canceled)

10. (canceled)

11. The method according to claim 1 wherein the KCC2 activator is combined with pharmaceutically acceptable excipients to form therapeutic composition.

12. The method according to claim 1 wherein prochlorperazine, CLP257, CLP290, a CLP257 derivative, or a CLP290 derivative is combined with pharmaceutically acceptable excipients to form therapeutic composition.
